SEQUENCE LISTING (12 sequences)

SEQ ID NO: 1 - DNA, 28 bp, Artificial Sequence
  sense primer for PCR of lipase gene
ggggatccgttggaaggagagggagaac

SEQ ID NO: 2 - DNA, 27 bp, Artificial Sequence
  antisense primer for PCR of lipase gene
gcggtacctttttgtccgttctcctga

SEQ ID NO: 3 - DNA, 29 bp, Artificial Sequence
  sense primer for PCR of cry1Aa promoter
tccccgcgggactcttcctatatttactt

SEQ ID NO: 4 - DNA, 20 bp, Artificial Sequence
  antisense primer for PCR of cry1Aa promoter
atttgtacaggaaatgcgtc

SEQ ID NO: 5 - DNA, 42 bp, Artificial Sequence
  sense primer for PCR of mutated CMCase gene
ggatccggggaggagaatcatgatctctatttttattacgtg

SEQ ID NO: 6 - DNA, 26 bp, Artificial Sequence
  antisense primer for PCR of mutated CMCase gene
gagctccagtatttcatccacaacgc

SEQ ID NO: 7 - DNA, 1491 bp, Artificial Sequence
  CMCase gene with mutated signal sequence to enhance its hydrophobicity
atgatctctatttttattacgtgtttattgattacgttattgacaatgggcggcatgctggcttcgccggcatcagcagcagggacaaaaacgccagtagccaagaatggccagcttagcataaaaggtacacagctcgttaaccgagacggtaaagcggtacagctgaaggggatcagttcacacggattgcaatggtatggagaatatgtcaataaagacagcttaaaatggctgagggacgattggggtatcaccgttttccgtgcagcgatgtatacggcagatggcggtataattgacaacccgtccgtgaaaaataaaatgaaagaagcggttgaagcggcaaaagagcttgggatatatgtcatcattgactggcatatcttaaatgacggtaatccaaaccaaaataaagagaaggcaaaagaattcttcaaggaaatgtcaagcctttacggaaacacgccaaacgtcatttatgaaattgcaaacgaaccaaacggtgatgtgaactggaagcgtgatattaaaccgtatgcggaagaagtgatttccgttatccgcaaaaatgatccagacaacattatcattgtcggaaccggtacatggagccaggatgtgaatgatgctgccgatgaccagctaaaagatgcaaacgttatggacgcacttcatttttatgccggcacacacggccaatttttacgggataaagcaaactatgcactcagcaaaggagcacctatttttgtgacagagtggggaacaagcgacgcgtctggcaatggcggtgtattccttgatcaatcgagggaatggctgaaatatctcgacagcaagaccatcagctgggtgaactggaatctttctgataagcaggaatcatcctcagctttaaagccgggggcatctaaaacaggcggctggcggttgtcagatttatctgcttcaggaacattcgttagagaaaacattctcggcaccaaagattcgacgaaggacattcctgaaacgccagcaaaagataaacccacacaggaaaacggtatttctgtacaatacagagcaggggatgggagtatgaacagcaaccaaatccgtccgcagcttcaaataaaaaataacggcaataccacggttgatttaaaagatgtcactgcccgttactggtataacgcgaaaaacaaaggccaaaacgttgactgtgactacgcgcagcttggatgcggcaatgtgacatacaagtttgtgacgttgcataaaccaaagcaaggtgcagatacctatctggaacttggatttaaaaacggaacgctggcaccgggagcaagcacagggaatattcagcttcgtcttcacaatgatgactggagcaattatgcacaaagcggcgattattcctttttcaaatcaaatacgtttaaaacaacgaaaaaaatcacattatatgatcaaggaaaactgatttggggaacagaaccaaattag

SEQ ID NO: 8 - protein, 496 aa, Artificial Sequence
  CMCase gene with mutated signal sequence to enhance its hydrophobicity
MISIFITCLLITLLTMGGMLASPASAAGTKTPVAKNGQLSIKGTQLVNRDGKAVQLKGISSHGLQWYGEYVNKDSLKWLRDDWGITVFRAAMYTADGGIIDNPSVKNKMKEAVEAAKELGIYVIIDWHILNDGNPNQNKEKAKEFFKEMSSLYGNTPNVIYEIANEPNGDVNWKRDIKPYAEEVISVIRKNDPDNIIIVGTGTWSQDVNDAADDQLKDANVMDALHFYAGTHGQFLRDKANYALSKGAPIFVTEWGTSDASGNGGVFLDQSREWLKYLDSKTISWVNWNLSDKQESSSALKPGASKTGGWRLSDLSASGTFVRENILGTKDSTKDIPETPAKDKPTQENGISVQYRAGDGSMNSNQIRPQLQIKNNGNTTVDLKDVTARYWYNAKNKGQNVDCDYAQLGCGNVTYKFVTLHKPKQGADTYLELGFKNGTLAPGASTGNIQLRLHNDDWSNYAQSGDYSFFKSNTFKTTKKITLYDQGKLIWGTEPN

SEQ ID NO: 9 - DNA, 58 bp, Artificial Sequence
  sense primer for PCR of mutated CMCase gene
ggatccggggaggagaatcatgcaccatcaccaccaccacgcagggacaaaaacgcca

SEQ ID NO: 10 - DNA, 26 bp, Artificial Sequence
  antisense primer for PCR of mutated CMCase gene
gagctccagtatttcatccacaacgc

SEQ ID NO: 11 - DNA, 1434 bp, Artificial Sequence
  CMCase gene with additional his encoding sequences
atgcaccatcaccaccaccacgcagggacaaaaacgccagtagccaagaatggccagcttagcataaaaggtacacagctcgttaaccgagacggtaaagcggtacagctgaaggggatcagttcacacggattgcaatggtatggagaatatgtcaataaagacagcttaaaatggctgagggacgattggggtatcaccgttttccgtgcagcgatgtatacggcagatggcggtataattgacaacccgtccgtgaaaaataaaatgaaagaagcggttgaagcggcaaaagagcttgggatatatgtcatcattgactggcatatcttaaatgacggtaatccaaaccaaaataaagagaaggcaaaagaattcttcaaggaaatgtcaagcctttacggaaacacgccaaacgtcatttatgaaattgcaaacgaaccaaacggtgatgtgaactggaagcgtgatattaaaccgtatgcggaagaagtgatttccgttatccgcaaaaatgatccagacaacattatcattgtcggaaccggtacatggagccaggatgtgaatgatgctgccgatgaccagctaaaagatgcaaacgttatggacgcacttcatttttatgccggcacacacggccaatttttacgggataaagcaaactatgcactcagcaaaggagcacctatttttgtgacagagtggggaacaagcgacgcgtctggcaatggcggtgtattccttgatcaatcgagggaatggctgaaatatctcgacagcaagaccatcagctgggtgaactggaatctttctgataagcaggaatcatcctcagctttaaagccgggggcatctaaaacaggcggctggcggttgtcagatttatctgcttcaggaacattcgttagagaaaacattctcggcaccaaagattcgacgaaggacattcctgaaacgccagcaaaagataaacccacacaggaaaacggtatttctgtacaatacagagcaggggatgggagtatgaacagcaaccaaatccgtccgcagcttcaaataaaaaataacggcaataccacggttgatttaaaagatgtcactgcccgttactggtataacgcgaaaaacaaaggccaaaacgttgactgtgactacgcgcagcttggatgcggcaatgtgacatacaagtttgtgacgttgcataaaccaaagcaaggtgcagatacctatctggaacttggatttaaaaacggaacgctggcaccgggagcaagcacagggaatattcagcttcgtcttcacaatgatgactggagcaattatgcacaaagcggcgattattcctttttcaaatcaaatacgtttaaaacaacgaaaaaaatcacattatatgatcaaggaaaactgatttggggaacagaaccaaattag

SEQ ID NO: 12 - protein, 477 aa, Artificial Sequence
  CMCase gene with additional his encoding sequences
MHHHHHHAGTKTPVAKNGQLSIKGTQLVNRDGKAVQLKGISSHGLQWYGEYVNKDSLKWLRDDWGITVFRAAMYTADGGIIDNPSVKNKMKEAVEAAKELGIYVIIDWHILNDGNPNQNKEKAKEFFKEMSSLYGNTPNVIYEIANEPNGDVNWKRDIKPYAEEVISVIRKNDPDNIIIVGTGTWSQDVNDAADDQLKDANVMDALHFYAGTHGQFLRDKANYALSKGAPIFVTEWGTSDASGNGGVFLDQSREWLKYLDSKTISWVNWNLSDKQESSSALKPGASKTGGWRLSDLSASGTFVRENILGTKDSTKDIPETPAKDKPTQENGISVQYRAGDGSMNSNQIRPQLQIKNNGNTTVDLKDVTARYWYNAKNKGQNVDCDYAQLGCGNVTYKFVTLHKPKQGADTYLELGFKNGTLAPGASTGNIQLRLHNDDWSNYAQSGDYSFFKSNTFKTTKKITLYDQGKLIWGTEPN

(19) United States
(12) Patent Application Publication
(10) Pub. No.: US 2004/0180348 A1
(43) Pub. Date: Sep. 16, 2004

(54) Method for surface display of proteins of genetic carriers

(76) Inventors: Jae-Gu Pan, Daejon-city (RU); Soo-Keun Choi, Sinsung-dong Yusung-gu (RU); Heung-Chae Jung, Daejon-City (RU)

Correspondence Address: NIXON & VANDERHYE, PC, 1100 N GLEBE ROAD, 8TH FLOOR, ARLINGTON, VA, 22201-4714, US

(21) Appl. No.: 10/466,208

(22) PCT Filed: Jan. 15, 2002
(86) PCT No.: PCT/KR02/00059

(30) Foreign Application Priority Data
Jan. 15, 2001 (KR) 2001/02156

(51) Int. Cl. C12Q 1/68; G01N 33/53; C12N 15/86

(52) U.S. Cl. 435/6; 435/7.1; 435/456; 435/69.1

(57) ABSTRACT

The present invention relates to methods for preparing a protein of interest surface-displayed on genetic carrier, for improving a protein of interest, for isolating a substance of interest, bioconversion and producing antibody. More particularly, the present invention relates to a method for preparing a protein of interest surface-displayed on genetic carrier, which comprises the steps of: (a) transforming a host cell harboring the genetic carrier selected from the group consisting of spore and virus with a vector containing a gene encoding the protein of interest; (b) culturing the transformed host cell and expressing the protein of interest in the host cell; and (c) allowing to form noncovalent bond between the expressed protein and a surface of the genetic carrier so that the protein of interest is displayed on the surface of the genetic carrier.

### TECHNICAL FIELD

The present invention relates to a method for surface display of proteins, in particular to methods for displaying a protein of interest on surface of spore, etc., for improving a protein of interest and for isolating a substance of interest.

### BACKGROUND ART

The technology of surface display in which organism displays on its surface the desired proteinaceous substance such as peptide and polypeptide has wider application fields depending on the types of protein displayed or host organism (Georgiou et al., 1993, 1997; Fischetti et al., 1993; and Schreuder et al., 1996). Such conventional surface display technology has been developed by use of several unicellular organisms such as bacteriophage, bacteria, yeast and mammalian cell.

The gene of protein to be displayed is contained in host organism and thus the host can be selectively screened using the characteristics of the protein displayed, thereby obtaining the desired gene from the selected host with easiness. Therefore, such surface display technology can guarantee a powerful tool on molecular evolution of protein (see WO 9849286; and U.S. Pat. No. 5,837,500).

High-Throughput Screening

For instance, phage displaying on its surface antibody having desired binding affinity is bound to immobilized antigen and then eluted, followed by propagating the eluted phage, thereby yielding the gene coding for target antibody from phage (U.S. Pat. No. 5,837,500). The bio panning method described above can provide a tool to select target antibody by surface displaying antibody library on phage surface in large amount and comprises the consecutive steps as follows: (1) constructing library; (2) surface displaying the library; (3) binding to immobilized antigen; (4) eluting the bound phage; finally (5) propagating selected clones.

The technology of phage surface display has been found to be useful in obtaining the desired monoclonal variant form enormous library (e.g., 10⁶-10⁹ variants) and thus applied to the field of high-throughput screening of antibody. Antibody has been used in various fields such as therapy, diagnosis, analysis, etc. and thus its demand has been largely increased. In this context, there has been a need for novel antibody to have binding affinity to new substance or catalyze biochemical reaction. The hybridoma technology to produce monoclonal antibody has been conventionally used so as to satisfy the need. However, the conventional method needs high expenditure and long time for performance whereas the yield of antibody is very low. In addition to this, to screen novel antibody, more than 10¹⁰ antibody libraries is generally used, as a result, the hybridoma technology has been thought to be inadequate in finding antibody exhibiting new binding property.

Many researches has focused on novel methods which is easier and more effective that the bio panning method described above and then developed novel technologies performed in such a manner that libraries are displayed on surface of bacteria or yeast and then cells displaying target protein is sorted with flow cytometry in a high-throughput manner. According to the technology, antigen labeled with fluorescent dye is bound to surface-displaying cell and the antibody having the desired binding affinity is isolated with flow cytometry capable of analyzing more than 10⁸ cells a hour. Francisco, et al., have demonstrated the usefulness of microbial display technology by revealing that surface-displayed monoclonal antibody could be concentrated with flow cytometry at rate of more than 10⁵, finally more than 79% have been proved to be the desired cells (Daugherty et al., 1998).

Live Vaccine

The surface display technology mentioned above can display antigen or fragment thereof and hence provide a delivery system for recombinant live vaccine. Up to now, attenuated pathogens or viruses have been predominantly employed as vaccine. Particularly, the bacteria have been found to express antigen intracellularly or extracellularly or on its cell membrane, thereby delivering antigen to host cell. The surface-displayed live vaccine induces a potential immune reaction and expresses continuously antigen during propagation in host cell; therefore, it has been highlighted as novel delivery system for vaccine. In particular, pathogen-derived antigenic epitope displayed on surface of nonpathogenic E. coli or Salmonella is administered orally in viable form and then exhibits to induce immune reaction in more continuous and powerful manner (Georgiou et al., 1997; and Lee et al., 2000).

Whole Cell Bioconversion

Whole cell as biocatalyst displaying on its surface enzyme capable of catalyzing chemical reaction can avoid necessities for direct expression, isolation and stabilization of enzyme. In case of expressing enzyme in cell for bioconversion, the cell is compelled to recovery and chemical (e.g., toluene) treatment to ensure impermeability of substrate. In addition, the lasting use renders the enzyme inactive or gives a problem on transference of substrate and product, thus dropping the productivity of overall process.

The above-mentioned shortcomings can be removed using enzyme displayed on cell surface (Jung et al, 1998a: 1998b). With whole cell displaying on its surface phosphodiesterase, organophosphorous-typed parathion and paraoxon with higher toxicity can be degraded, which is a typical example representing the applicability of cells displaying enzyme to environmental purification process (Richins et al., 1997).

Antipeptide Antibody

Martineau et al. have reported a highly simple method for production of antipeptide antibody using surface display technology of E. coli (Martineau et al., 1991). As described, the desired peptide is displayed on the protruding region of MalE and outer membrane protein, LamB and then whole cell or fragmented cell is administered to animal so as to generate antipeptide antibody. The method makes it possible to produce antibody with avoiding chemical synthesis of peptide and its linkage to carrier protein.

Whole Cell Absorber

To immobilize antibody or polypeptide on suitable carrier, which is useful in absorption chromatography, several subsequent steps must be performed, for example, protein production by fermentation, isolation of protein in pure form, and immobilization on a carrier. Generally, it is difficult to prepare the bioabsorber.

As absorber, a whole cell displaying absorption protein has been developed. The whole cell absorber known mostly is Staphylococcus aureus displaying on its surface protein A naturally, which has a high binding affinity to Fc domain of mammalian antibody. Currently, novel method has been proposed to remove and recover heavy metals, which employs metallothionein or metal-absorption protein displayed on microbial cell surface in large amount (Sousa et al., 1996, 1998; and Samuelson et al., 2000). The method is more effective in removing and recovering heavy metals from contamination source in comparison with the conventional method using metal-absorption microbes.

As understood based on the matters described above, in order to display foreign protein on cell surface, a suitable surface protein and foreign protein must be linked each other in gene level to express fusion protein, and the fusion protein should pass stably across inner membrane of cell to be attached to cell surface. Preferably, the surface protein having the following characteristics is recommended as surface display motif: 1) existence of secretory signal enabling passage across inner membrane of cell, 2) existence of target signal enabling stable attachment to cell surface, 3) high expression level on cell surface, and 4) stable expression regardless of protein size (Georgiou et al., 1993).

Meanwhile, according to the existing surface display methods described above, the motif for surface display is required to genetically modified in order to incorporate a protein of interest to N- or C-terminal, or central region of surface protein. All the proteins surface-displayed is expressed in a fusion form with surface display motif. Therefore, the resulting protein surface-displayed is a modified protein rather than wild type protein.

Up to date, the developed surface display systems are as follows: phage surface display system (Chiswell and McCarferty, 1992), bacterial surface display system (Georgiou et al., 1993; Little et al., 1993; and Georgiou et al., 1997), surface display system of Gram negative bacteria (Francisco et al., 1992; Fuchs et al., 1991; Klauser et al., 1990, 1992; and Hedegaard et al., 1989), surface display system of Gram positive bacteria (Samuelson et al., 1995; Palva et al., 1994; and Sleytr and Sara, 1997), and surface display system of yeast (Ferguson, 1988; and Schreuder et al., 1996). Furthermore, it has been developed that a protein of interest fused to spore coat protein is displayed on spore surface. For example, U.S. Pat. No. 5,766,914 discloses a method of producing and purifying enzymes using fusion protein between cotC or cotD among spore coat proteins of Bacillus subtilis and lacZ as reporter. U.S. Pat. Nos. 5,837,500 and 5,800,821 also indicate cotC and cotD as a preferable surface display motif, but its experimental demonstrations are not described.

According to a surface display system of Gram negative bacteria, the incorporation of foreign polypeptide into surface structure results in not only its steric limitation which makes it impossible to have stable membrane protein (Charbit et al., J. Immunol, 139:1644-1658(1987); and Agterberg et al., Gene, 88:37-45(1990)) but also drop of the stability of cell outer membrane and its viability. E. coli as display host, which has been intensively studied, uses generally cell outer membrane protein as surface display motif. However, the over-expression of cell outer membrane protein fused to foreign protein is likely to bring about structural instability of cell outer membrane, consequently, diving the viability of host cell (Georgiou et al., 1996).

The problems in the conventional display methods described above, is due to preparation of fusion protein between a protein of interest and surface display motif for display. Where the fusion protein is expressed in small amount, the reaction efficiency in whole cell bioconversion, protein array and antibody production is decreased; if overexpressed, it is very likely to lead to the shortcomings mentioned above. In addition, the surface display methods using the fusion protein are depended on the extent of incorporation of surface display motif into cell, spore or phage surface, giving rise to limitation of the amount of protein displayed.

As described above, the conventional surface display technology is fundamentally dependent on formation of fusion protein between a protein of interest and surface display motif. Consequently, there occur several shortcomings in conventional surface display systems: (1) necessity of getting knowledge of a gene sequence of surface display motif; (2) necessity of cloning a gene of surface display motif; (3) being very likely to affect the tertiary structure of a protein of interest by surface display motif; (4) rendering a protein of interest inactive when a protein of interest is active only in multimeric form and a fusion protein is independently surface-displayed; (5) limitation of the amount of protein displayed since the surface display methods using the fusion protein are depended on the extent of incorporation of surface display motif into host cell surface; (6) inducing a structural instability of host cell surface when a protein of interest is surface displayed in excess, thereby dropping resistance to environment and viability of host cell.

Consequently, for developing novel surface display system, which is capable of overcoming the shortcomings in conventional methods, the following characteristics should be accomplished: (1) being capable of constructing system without knowledge on a gene sequence of surface display motif; (2) being capable of constructing system without cloning a gene of surface display motif; (3) being capable of displaying a protein of interest on host cell surface after forming a inherent structure thereof; (4) being capable of increasing an amount of protein surface-displayed by means of nonselective linkages; and/or (6) not reducing a resistance to environment and a viability of host cell even when a protein of interest is surface displayed in excess.

Throughout this application, various patents and publications are referenced and citations are provided in parentheses. The disclosure of these patents and publications in their entities are hereby incorporated by references into this application in order to more fully describe this invention and the state of the art to which this invention pertains.

### DISCLOSURE OF INVENTION

Under such situation, the present inventors have made intensive studies to be from the shortcomings of the conventional surface display methods, and as a result, we have developed novel display system eliminating the need of motif for surface display. Surprisingly, it has been found that the developed system is capable of displaying any protein on surface with maintaining inherent structure thereof and when displaying in excess, a genetic carrier maintains its viability and resistance to surrounding environment.

Accordingly, it is an object of this invention to provide a method for preparing a protein of interest surface-displayed on genetic carrier.

It is another object of this invention to provide a method for improving a protein of interest by using the method for surface display on genetic carrier.

It is still another object of this invention to provide a method for isolating a substance of interest in mixture by using the method for surface display on genetic carrier.

It is further object of this invention to provide a method for bioconversion by using the method for surface display on genetic carrier.

It is still further object of this invention to provide a method producing an antibody to antigen in vertebrates by using the method for surface display on genetic carrier.

It is another object of this invention to provide a vector for displaying on surface of genetic carrier a protein of interest.

It is still another object of this invention to provide a microbial transformant for displaying on surface of genetic carrier a protein of interest.

It is further object of this invention to provide a complex between genetic carrier and protein of interest.

It is still further object of this invention to provide a genetic carrier library diaplaying on its surface variants of a protein of interest.

It is another object of this invention to provide a protein microarray prepared by using the method for surface display on genetic carrier.

### BRIEF DESCRIPTION OF THE DRAWINGS

FIG. 1 schematically illustrates the principle of the present invention;

FIG. 2 is a graph representing activity of lipase of Pseudomonas fluorescens which is displayed on spore surface;

FIG. 3 is a graph demonstrating spore-surface display of wild type lipase which is expressed in host cell;

FIG. 4 is a graph showing the result of flow cytometry analysis for confirming spore-surface display of wild type carboxymethylcellulase;

FIG. 5 is a genetic map of vector, pCry1p-CMCase-hp, for spore-surface display;

FIG. 6 is a graph showing the result of flow cytometry analysis for confirming spore-surface display of carboxymethylcellulase with modified secretory signal;

FIG. 7 is a genetic map of vector, pCry1p-CMCase-his, for spore-surface display; and

FIG. 8 is a graph showing the result of flow cytometry analysis for confirming spore-surface display of carboxymethylcellulase with a fusion sequence, cationic domain.

### BEST MODE FOR CARRYING OUT THE INVENTION

The term used firstly herein, “genetic carrier” refers to an organism displaying on its surface a protein of interest and having the following properties: (1) selected from the group consisting of spore and virus; (2) having capacity of forming noncovalent bond to a protein of interest with a desired dissociation constant, expressed in host cell harboring the genetic carrier; and (3) if necessary, its surface properties is able to be modified via genetic engineering method.

The term used herein “host cell” has a different meaning from one disclosed and indicated in prior publications related to surface display of protein. The term used herein “host cell” refers to a cell expressing a protein of interest and having the following properties: (1) being capable of being transformed with a gene encoding a protein of interest; (2) being capable of harboring genetic carrier such as spore and virus and proliferating the genetic carrier; and (3) being capable of being manipulated genetically, if necessary.

As described above, in the present specification, the terms, “genetic carrier” displaying on its surface a protein of interest and “host cell” expressing a protein of interest are employed with strictly different meanings.

In one aspect of this invention, there is provided a method for preparing a protein of interest surface-displayed on genetic carrier, which comprises the steps of: (a) transforming a host cell harboring the genetic carrier selected from the group consisting of spore and virus with a vector containing a gene encoding the protein of interest; (b) culturing the transformed host cell and expressing the protein of interest in the host cell; and (c) allowing to form noncovalent bond between the expressed protein and a surface of the genetic carrier so that the protein of interest is displayed on the surface of the genetic carrier.

In another aspect of this invention, there is provided a method for improving a protein of interest, which comprises the steps of: (a) constructing a gene library of the protein of interest by means of mutating the gene encoding the protein of interest; (b) preparing a vector library containing the constructed gene library; (c) transforming a host cell harboring a genetic carrier selected from the group consisting of spore and virus with the vector library; (d) culturing the transformed host cell and expressing the variants of the protein of interest in the host cell; (e) obtaining a genetic carrier library by means of allowing to form noncovalent bond between the expressed protein variant and a surface of the genetic carrier so that the variant is displayed on the surface of the genetic carrier; and (f) screening the genetic carrier displaying on its surface the variant of the protein of interest having a desired property.

In still another aspect of this invention, there is provided a method for isolating a substance of interest in mixture, which comprises the steps of: (a) constructing a gene library encoding a variant of binding protein or binding domain thereof by means of mutating the gene encoding the binding protein or binding domain as protein of interest; (b) preparing a vector library containing the constructed gene library; (c) transforming a host cell harboring a genetic carrier selected from the group consisting of spore and virus with the vector library; (d) culturing the transformed host cell and expressing the variants of the binding protein or binding domain in the host cell; (e) obtaining a genetic carrier library by means of allowing to form noncovalent bond between the expressed binding protein variant or binding domain variant and a surface of the genetic carrier so that the variant is displayed on the surface of the genetic carrier; (f) contacting the genetic carrier library with a predetermined substance and screening an improved binding protein or binding domain thereof by means of selecting the genetic carrier displaying on its surface the variant binding the predetermined substance; (g) contacting the genetic carrier displaying on its surface the improved binding protein or binding domain thereof with the mixture to isolate the substance of interest in mixture.

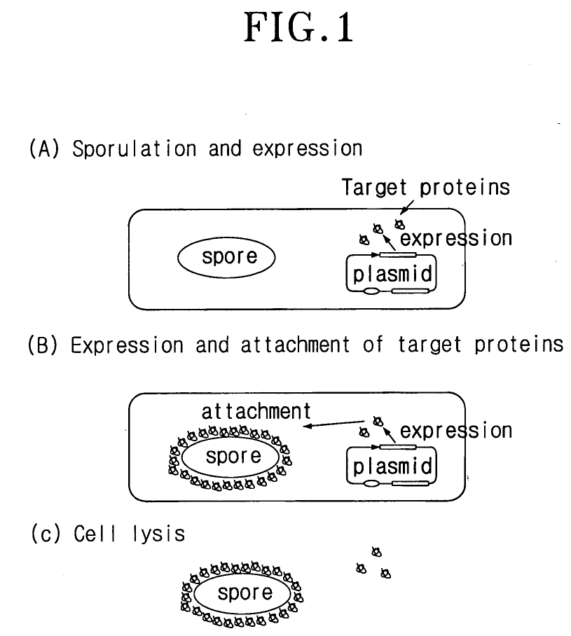

The present method has been developed based on a novel concept, which is largely different from the conventional surface display methods. The present method takes advantage of properties of constituents on surface of genetic carrier and, in particular, noncovalent bonds between a protein on surface of genetic carrier and a protein of interest. The principle strategy of this invention, using a spore as genetic carrier, is illustratively exemplified in FIG. 1. Referring to FIG. 1, a host cell is transformed with vector carrying a sequence encoding a protein of interest, the protein of interest is expressed intracellularly or extracellularly at or prior the period of forming spore and the surface display of protein of interest is finally accomplished by virtue of noncovalent bonds between the protein of interest and the surface of spores formed in host cell.

As described above, the striking feature of the present invention lies in eliminating a need of a motif for surface display which is essential in conventional methods for surface display of protein. Because the instant method circumvents a necessity for a motif for surface display, the proteins found to be difficult to pass across cell membrane, when expressed in host cell, can be displayed well on surface of genetic carrier and when host cells are lysed to expose the genetic carrier, the genetic carriers displaying on its surface the proteins can be recovered. The recovered complex between protein of interest and genetic carrier has a wide application.

According to the present methods, a spore or virus can be employed as genetic carrier. The spore is a preferable genetic carrier due to its properties as follows (Driks, 1999): (1) a higher heat stability; (2) a significant stability to radioactivity; (3) a stability to toxins; (4) a higher stability to acid and base; (5) a significant stability to lysozyme; (6) a resistance to dryness; (7) a higher stability to organic solvents; (8) no metabolic activity; and (9) shorter time for obtaining spore, e.g. within several hours.

According to the methods, when virus is used as genetic carrier, it is preferred to use bacteriophage, and the protein of interest expressed in prokaryotic host cell is surface-displayed via nonconvalent bond to coat proteins of the bacteriophage. Where the bacteriophage is located in periplasm of host cell, the signal peptide may be fused to the protein of interest to permit secretion toward periplasm, thereby ensuring a surface display. If the protein of interest cannot be naturally bound to coat proteins of bacteriophage, it may be fused to a motif capable of binding to coat proteins of bacteriophage in order to allow surface display.

According to a preferred embodiment, the genetic carrier has a surface protein modified to enhance noncovalent bond with the protein of interest. The method for modification of the genetic carrier includes: (i) fusing oligopeptide or polypeptide, which enhance noncovalent bond between the protein of interest and genetic carrier, to the surface protein of genetic carrier; (ii) subjecting the surface protein of genetic carrier to site-directed mutagenesis; and (iii) subjecting the surface protein of genetic carrier to random mutagenesis, but not limited to.

In the present methods, the protein of interest includes any protein and peptide, for example, hormone, hormone analogue, enzyme, enzyme inhibitor, signal transduction protein or fragment thereof, antibody or fragment thereof, single chain antibody, binding protein or fragment thereof, peptide, antigen, adhesive protein, structural protein, regulatory protein, toxin protein, cytokine, transcription regulatory protein, blood clotting protein and plant defense-inducing protein, but not limited to.

The binding protein or binding domain thereof used in this invention, includes any protein or domain thereof capable of a predetermined substance, for example, antibody or antibody domain, when a certain antigenic substance is isolated. The binding protein or binding domain includes, but not limited to, protease inhibitor, crambin, enterotoxin, conotoxin, apaminm lysozyme, ribonuclease, charybdotoxin, cystatin, eglin, ovomucoid, azurin, tumor necrosis factor and CD4.

According to the present methods, either monomer or multimer (including homo multimer and hetero multimer) can be surface-displayed. Multimeric protein has generally a complete activity only when all of its monomers are combined. In conventional methods, it has been found that multimeric protein is surface-displayed in inactive form because its monomers are independently surface-displayed each other. According to the present methods, the multimeric proteins can be displayed on surface of genetic carrier with maintaining its integral structure.

According to a preferred embodiment, the protein of interest to be surface-displayed may be modified so as to enhance noncovalent bonds to genetic carrier. The modification methods include: (i) deleting a portion of amino acids of the protein of interest; (ii) fusing oligopeptide or polypeptide, which enhance noncovalent bond between the protein of interest and genetic carrier, to the protein of interest or deleted form of (i); (iii) subjecting the protein of interest to site-directed mutagenesis; and (iv) subjecting the protein of interest to random mutagenesis, but not limited to. The method of deleting a portion of amino acids of the protein of interest may be performed in various manners, for example, by deleting ionic amino acids from N-terminal sequence (e.g. signal peptide) of the protein of interest. The protein of interest thus modified enhances hydrophobic interaction with genetic carrier and therefore, can be surface-displayed with lower dissociation constant. It has been reported that the spore surface carries anionic charge. Therefore, it is preferred that a cationic peptide is fused to the protein of interest for surface display.

In the present methods, as a gene encoding protein of interest to be transformed, two or more repeated sequences are also useful. In two or more repeated sequences, the repeated sequences may be the same or different each other. Other combinations also may be useful in the fusion sequence. In addition, the gene used in transformation, may exist as plasmid in host cell independently or as integrated form into chromosome of host cell.

The expression of protein of interest can be induced by virtue of its own promoter or other suitable promoters inducible in host cell.

According to the present methods, noncovalent bonds, in particular, one or more among hydrophobic bond, ionic bond, hydrogen bond, and van der Waals bond, permits the interaction between protein of interest and genetic carrier.

According to a preferred embodiment, the host cell harboring spore includes, but not limited to, a spore-forming Gram negative bacterium such as Myxococcus; a spore-forming Gram positive bacterium such as Clostridium, Paenibacillus and Bacillus; a spore-forming Actionmycete; a spore-forming yeast such as Saccharomyces cerevisiae, Candida and Hansenulla and a spore-forming fungus. More preferably, the host cell is the spore-forming Gram positive bacterium and the most preferably, Bacillus including Bacillus subtilis, Bacillus thuringiensis and Bacillus megaterium. In particular, Bacillus subtilis is advantageous in the senses that genetic knowledge and experimental methods on its spore forming as well as culturing method are well known.

According to a preferred embodiment, the host cell is one mutated to eliminate a production of intracellular protease or extracellular protease which is involved in degradation of the surface-displayed protein of interest.

Although the present methods is fundamentally directed to surface display via noncovalent bond between genetic carrier and protein of interest, additional covalent bond may be used, if necessary for more stabilized linkage. The stabilizing the bond between the surface of the genetic carrier and the protein of interest can be performed by means of forming covalent bonds to between the surface of the genetic carrier and the protein of interest by use of physical, chemical or biochemical methods following displaying the protein of interest on the surface of genetic carrier via noncovalent bond. Among the methods to form covalent bond, a treatment of glutaraldehyde (DeSantis G. and Jones J. B. Curr. Opin. Biotechnol. 10:324-330(1999)) is preferable as chemical method, a treatment of ultraviolet (Graham L., and Gallop P. M. Anal. Biochem. 217:298-305(1994)) is preferable as physical method and a treatment of enzyme ensuring formation of covalent bond (Gao Y., and Mehta K., J. Biochem. 129:179-183(2001)) as biochemical method.

In the method for preparing a protein of interest surface-displayed on genetic carrier, it is preferred that the method further comprises the step of screening the genetic carrier displaying on its surface the protein of interest.

In the method for improving a protein of interest, the step of constructing a gene library by mutation of wild type gene of protein of interest by means of: DNA shuffling method (Stemmer, Nature, 370: 389-391(1994)), StEP method (Zhao, H., et al., Nat. Biotechnol., 16: 258-261 (1998)), RPR method (Shao, Z., et al., Nucleic acids Res., 26: 681-683 (1998)), molecular breeding method (Ness, J. E., et al., Nat. Biotechnol., 17: 893-896 (1999)), ITCHY method (Lutz S. and Benkovic S., Current Opinion in Biotechnology, 11: 319-324 (2000)), error prone PCR (Cadwell, R. C. and Joyce, G. F., PCR Methods Appl., 2: 28-33 (1992)) and point mutagenesis (Sambrook et al., Molecular Cloning: A Laboratory Manual, Cold Spring Harbor, N. Y., 1989).

According to a preferred embodiment of method for improving a protein of interest, the genetic carrier is spore and the step of screening is performed in such a manner that the spore library is treated with one or more selected from the group consisting of organic solvent, heat, acid, base, oxidant, dryness, surfactant and protease and then spore displaying on its surface the variant of protein of interest resistant to the treatment is selected.

According to another preferred embodiment of method for improving a protein of interest, the genetic carrier is spore and the step of screening is performed in such a manner that the spore library is primarily treated with one or more selected from the group consisting of organic solvent, heat, acid, base, oxidant, dryness and surfactant followed by secondary treatment with protease and then spore displaying on its surface the variant of protein of interest resistant to the protease is selected.

In the present methods, the step of screening may be performed by means of: (i) an activity of the protein of interest displayed on surface of genetic carrier; (ii) a protein being capable of recognizing a substance labeling the protein of interest; (iii) a labeled ligand being capable of binding to the protein of interest; or (iv) an antibody being capable of binding to the protein of interest specifically, but not limited to. Preferably, flow cytometry is employed in the screening by means of a labeled ligand being capable of binding to the protein of interest or an antibody being capable of binding to the protein of interest specifically. For example, a primary antibody is bound to the protein of interest displayed on spore surface and then reacted with a secondary antibody labeled with fluorescent chemical to stain the spore, followed by observation with fluorescence microscope or analysis with flow cytometry. If the secondary antibody is labeled with gold, the observation with electron microscope is enabled. Screening by use of activity of protein of interest can be performed by measuring colorimetric reaction catalyzed by the protein.

In the present methods for preparing a protein of interest surface-displayed and for improving a protein of interest, it is preferred that following proliferation of genetic carrier screened, the protein variants with desired properties or genes encoding them are recovered.

According a preferred embodiment using spore as genetic carrier, the recovery of spore is performed in such a manner that the display of the protein of interest on the spore surface is maximized by controlling culture time, after which culturing is terminated and the spore is then recovered. Suitable culture time is varied depending upon the type of cell used, for example, in case of using Bacillus subtilis as host, the culture time of 16-25 hours is preferred. The recovery of spore may be carried out according to the conventional methods known to one skilled in the art, more preferably, renografin gradients methods (C. R. Harwood, et al., “Molecular Biological Methods for Bacillus.” John Wiley & Sons, New York, p. 416 (1990)).

The method for improving protein provide in a high-throughput manner, from wild type, (1) enzymes catalyzing non-biological reaction (e.g., Diels-Alder condensation); (2) enzymes with non-natural steroselectivity or regioselectivity; (3) enzymes with activity in organic solvent or organic solvent-aqueous solution two-phase system; and (4) enzymes with activity in extreme conditions such as high temperature or pressure. In addition, to select a variant of antibody with enhanced binding affinity, it is general that pH is abruptly changed or the concentration of base is adjusted to elute the variant. In a method using phage or bacteria as carrier, such elution conditions are likely to decrease the viability of phage or bacteria in medium. However, the methods for improving protein using system of spore surface display overcome the drawback.

In further aspect of this invention, there is provided a vector for displaying on surface of genetic carrier a protein of interest, which comprises a replication origin, an antibiotic-resistance gene, a restriction site, a gene encoding the protein of interest, wherein the protein of interest, when expressed in host cell, is capable of forming noncovalent bond to the surface of genetic carrier. According to a preferred embodiment, the gene encoding the protein of interest is one mutated to enhance noncovalent bond between the surface of genetic carrier and the protein of interest. The gene encoding the protein of interest is mutated to (i) delete a portion of amino acids of the protein of interest; (ii) fuse oligopeptide or polypeptide, which enhance noncovalent bond between the protein of interest and genetic carrier, to the protein of interest or deleted form of (i); (iii) subject the protein of interest to site-directed mutagenesis; or (iv) subject the protein of interest to random mutagenesis.

In still further aspect of this invention, there is provided a microbial transformant, characterized in that the transformant is produced by transformation a host cell harboring spore or virus with the present vector. According to a preferred embodiment, the host cell is one mutated to eliminate a production of intracellular protease or extracellular protease which is involve in degradation of the surface-displayed protein of interest.

In another aspect of this invention, there is provided a complex between genetic carrier and protein of interest, characterized in that the complex is prepared by displaying on the surface of the genetic carrier, according to the method of claim 1, hormone, hormone analogue, enzyme, enzyme inhibitor, signal transduction protein or fragment thereof, antibody or fragment thereof, single chain antibody, binding protein or fragment thereof, peptide, antigen, adhesive protein, structural protein, regulatory protein, toxin protein, cytokine, transcription regulatory protein, blood clotting protein or plant defense-inducing protein.

According to a preferred embodiment, the protein of interest is one modified by virtue of: (i) deleting a portion of amino acids of the protein of interest; (ii) fusing oligopeptide or polypeptide, which enhance noncovalent bond between the protein of interest and genetic carrier, to the protein of interest or deleted form of (i); (iii) subjecting the protein of interest to site-directed mutagenesis; or (iv) subjecting the protein of interest to random mutagenesis.

In addition, the present complex may have additional covalent bonds to stabilize the bond between the surface of the genetic carrier and the protein of interest, in which the covalent bonds are formed by use of physical, chemical or biochemical methods following displaying the protein of interest on the surface of genetic carrier via noncovalent bond.

In the present complex, a spore is a preferable genetic carrier. Where a spore is used as genetic carrier, it is preferred that the spore is non-reproductive one obtained by means of one or more methods selected from the group consisting of genetic method (Popham D. L., et al., J. Bacteriol., 181: 6205-6209 (1999)), chemical method (Setlow T. R., et al., J. Appl. Microbiol., 89: 330-338 (2000)) and physical method (Munakata N, et al., Photochem. Photobiol., 54: 761-768 (1991)). The present complex using a spore only as display means of protein of interest can obviate the necessity for reproduction of spore. It is considerable that the organisms genetically engineered is likely to be regulated under laws and rules; hence non-reproductive spore is preferable. The genetic method for rendering spore non-reproductive may be carried out by deletion of gene involved in spore reproduction of host cell. For example, Bacillus subtilis lack of cwlD gene is preferably used in this invention. Furthermore, it is preferable that the spore is derived from a variant mutated to increase its agglutination property by one or more methods selected from the group consisting of physical method (Wienc K. M., et al., Appl. Environ. Microbiol., 56: 2600-2605 (1990)), chemical method and genetic method. The spore with increased agglutination property is conveniently separated from resulting product in bioconversion performed in industrial scale.

In a preferred embodiment, the genetic carrier is a bacteriophage.

In still another aspect of this invention, there is provided a genetic carrier library diaplaying on its surface variants of a protein of interest, prepared by a process comprising the steps of: (a) constructing a gene library of the protein of interest by means of mutating the gene encoding the protein of interest; (b) preparing a vector library containing the constructed gene library; (c) transforming a host cell harboring a genetic carrier selected from the group consisting of spore and virus with the vector library; (d) culturing the transformed host cell and expressing the variants of the protein of interest in the host cell; (e) obtaining a genetic carrier library by means of allowing to form noncovalent bond between the expressed protein variant and a surface of the genetic carrier so that the variant is displayed on the surface of the genetic carrier; and (f) screening the genetic carrier displaying on its surface the variant of the protein of interest having a desired property.

According to a preferred embodiment, the genetic carrier is a spore or bacteriophage.

In further aspect of this invention, there is provided a method for bioconversion using protein with activity for conversion reaction, characterized in that the method employs the present complex between genetic carrier and protein of interest. Any protein capable of catalyzing (bio)chemical reaction including enzyme and enzymatic antibody is useful in this bioconversion.

Meanwhile, the bioconversion process using surface-displayed enzymes requires a physiochemical stability of surface displaying genetic carrier in extreme conditions because the process is usually executed in high temperature and/or organic solvent. In particular, a chemical synthesis valuable in current industry is mainly carried out in organic solvent and the synthesis of chiral compound or the resolution of racemic mixture is also performed in highly severe physiochemical conditions. Therefore, the surface-displayed enzyme as well as the organisms displaying enzyme is compelled to have stability in such extreme conditions. In this connection, it is demonstrated that the present method for bioconversion using spore or virus for surface display is largely advantageous.

The chemical processes using surface-displayed enzymes have been proposed (Georgiou et al., 1993). However, the proposed processes have generally required immobilization of cell surface with cross-linking agent since the host displaying enzyme is very unstable during process (Freeman et al., 1996) The present bioconversion process can be free from the disadvantage mentioned above. Because the surface-displayed enzyme as well as the genetic carrier displaying enzyme is largely stable, the present method avoids the immobilization. The present bioconversion method can be also applied to any type of enzyme such as lipase, protease, cellulase, glycosyltransferase, oxidoreductase and aldolase. In addition, the present method is useful in single step or multi-step reaction and in aqueous or non-aqueous solution. The present bioconversion method employs genetic carrier as free or immobilized form and can be performed with other microbes or enzymes.

In still further aspect of this invention, there is provided a method producing an antibody to antigen in vertebrates, characterized in that the method comprises administering to vertebrates a composition containing an immunologically effective amount of the present complex between genetic carrier and protein of interest. According to the method producing an antibody of this invention, a composition containing an immunologically effective amount of the complex, preferably, further comprises adjuvant such as incomplete and complete Freund's adjuvants. In the present method, the administration may be carried out by oral and intravenous, intraperitoneal, subcutaneous and intramuscular injections. Boosting within suitable period after the first administration is preferable to yield a sufficient amount of antibody.

Similar to DNA microarray, a protein microarray provides means for analyzing expression or expression level of target protein in certain cell. In order to fabricate protein array, the suitable proteins to be arrayed must be obtained and then immobilized on solid surface. During analysis using protein array, washing step is necessarily performed to remove unbound proteins and various treatments such as high temperature, higher salt concentration and pH adjustment are executed; therefore, it is pivotal to guarantee proteinaceous substance with higher stability in such detrimental environment. In addition, the conventional process for preparing protein array needs tedious and repetitive works such as cloning genes of several thousands to tens of thousands of proteins and immobilizing of the proteins expressed. Therefore, there remains a need to improve simplicity and rapidity of the works.

According to the method for preparing protein microarray of this invention, it is ensured that the works described-above cane be performed with much greater readiness. In the present method, the present complex or the genetic carrier library aforedescribed is immobilized onto the solid substrate. The present protein microarray is prepared by means of immobilization of genetic carrier, which displays on its surface a protein of interest, onto a solid substrate. In the method for preparing protein array, the conventional processes may be used (see WO 0061806, WO 0054046, U.S. Pat. No. 5,807,754, EP 0818467, WO 9742507, U.S. Pat. No. 5,114,674 and WO 9635953). The protein microarray manufactured by the present invention has a variety of applicable fields including diagnosis, analysis of gene expression, analysis of interaction between proteins, analysis of interaction between protein and ligand, study on metabolism, screening novel or improved enzymes, combinatorial biochemical synthesis and biosensor.

The solid substrate suitable in the present method includes, but not limited to, glasses (e.g., functionalized glasses), Si, Ge, GaAs, GaP, SiO, SiN₄, modified silicone nitrocellulose, polyvinylidene fluoride, polystylene, polytetrafluoroethylene, polycarbonate, nylon, fiber and combinations thereof. The genetic carrier optionally may be attached to the array substrate through linker molecules. It is preferred that the regions of the array surface not being spotted are blocked. The amount of genetic carrier applied to each spot (or address) depends on the type of array. Interaction between the protein displayed on genetic carrier attached to solid substrate and the sample applied can be detected based on their inherent characteristics (e.g., immunogenicity) or can be rendered detectable by being labeled with an independently detectable tag (e.g., fluorescent, luminescent or radioactive molecules, and epitopes) . The data generated with protein array of this invention can be analyzed using known computerized systems such as “reader” and “scanner”.

As understood from the all descriptions in this application, the present surface display method using genetic carrier adapted to all the present methods has several advantages: (1) avoiding a need of a motif for surface display; (2) ensuring a protein of interest to have its intact activity in such a manner that the protein of interest forms its inherent structure following expression and then is displayed on surface; (3) increasing an amount of a protein of interest surface-displayed because the protein is displayed via noncovalent bonds, i.e., in non-selective manner; and (4) no or little effect on viability and resistance to surrounding environment of genetic carrier although an amount of the protein surface-displayed is increased.

The following specific examples are intended to be illustrative of the invention and should not be construed as limiting the scope of the invention as defined by appended claims.

### EXAMPLES

Example 1

### Validation on Spore Surface Display of Pure Isolated Lipase

It has remained to be verified that a cytoplasmic protein can be bound and displayed on the spore surface as previously reported coat protein or structural protein (morphogen). On the base of the hydrophobic property of spore surface (Wiencek, K. M. et al., Appl. Environ. Microbiol., 56:2600-2605 (1990)), the present inventors hypothesized that proteins with hydrophobic domains e.g. lipase (Brockerhoff H., Chem. Phys. Lipids, 10:215 (1973)) may be attached to and thus displayed on the spore surface via hydrophobic bond. The hypothesis was verified by measuring the enzymatic activity of lipase after attachment of lipase, which is purified from Pseudomonas fluorescens, on pure spore isolated from Bacillus subtilis.

Firstly, Bacillus substilis DB104 strain (Kawamura F. and Doi R. H., J. Bacteriol., 160:442-444(1984)) was cultured for 24 hr at a shaking incubator (37□, 250 rpm) in GYS medium ((NH₄)₂SO₄ 2 g/l, yeast extract 2 g/l, K₂HPO₄ 0.5 g/l, glucose 1 g/l, MgSO₄.H₂O 0.41 g/l, CaCl₂.2H₂O 0.08 g/l, MnSO₄.5H₂O 0.07 g/l ), and the only pure spores were isolated using renografin gradients method (C. R. Harwood, et al., “Molecular Biological Methods for Bacillus.” John Wiley & Sons, New York, p.416(1990)). The pure isolated spores were confirmed under a microscope (1000×, ALPHAPHOT-2, Nikon).

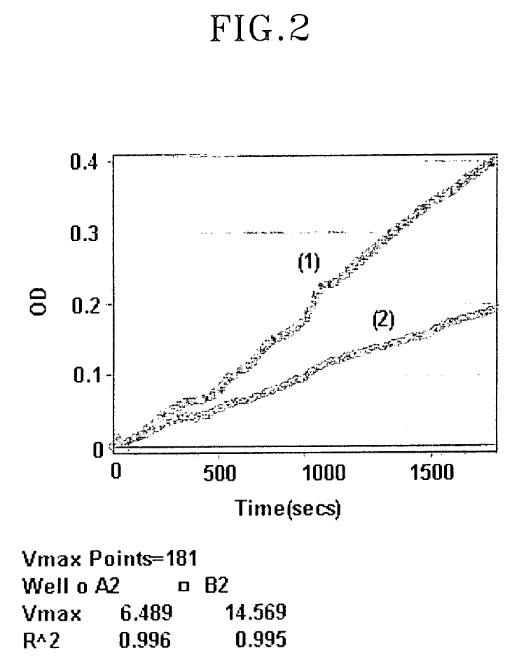

2 mg of the pure isolated Bacillus spores and 94□ of the partially purified Pseudomonas lipases (Ahn, J. H. et al., J. Bacteriol., 181:1847-1852(1999)) were mixed into 200 l of 50 mM Tris buffer (pH 8.0), reacted without disturbance for 12 hr at 4□ and the spores were isolated from the buffer by centrifugation. Subsequently, the isolated spores were rinsed three times with 0.5□ of 50 mM Tris buffer (pH 8.0) and finally the lipase-attached spores were purified. To measure the lipase activity attached on spores, the lipase-attached spores were suspended into PBS buffer, added 10% olive oil, reacted for 48 hr, 0.2□ cupric acid was treated on supernatant, and the final OD was measured at 715 nm. The result of supernatant indicated the lipase activity released from lipase-attached spores by olive oil (FIG. 2). In FIG. 2, the line (1) represents the lipase-attached spores and the line (2) is control without lipase attachment, and the horizontal line refers to the reacting time for lipase activity.

These results indicate that the lipase can bind to spore surface only via absorption due to hydrophobic interaction.

These results exemplifies that any protein with hydrophobicity can be displayed on spore, and it is understood for those skilled in the art that a protein with hydrophobicity can be displayed on spore surface when the protein is expressed in a cell or secreted out of a cell.

### Example 2

### Display of Wild Type Lipase on Spore Surface

The spore display of wild type lipases, which are expressed in host cells, is examined as follows: The plasmid pBS:lipA (Bell P. J. L. et al, Biotechnol. Lett., 21:1003-1006(1999)) was gifted by Dr. Bergquist in Australia. PCR was performed using primer lip1 (SEQ ID No:1) and primer lip2 (SEQ ID No:2) with template of the pBS:lipA plasmid. Taq polymerase purchased from Boehringer Mannheim was used for total 35 cycles of PCR under condition of denaturation for 30 sec at 94□, annealing for 30 sec at 55□ and extension for 1 min at 72□.

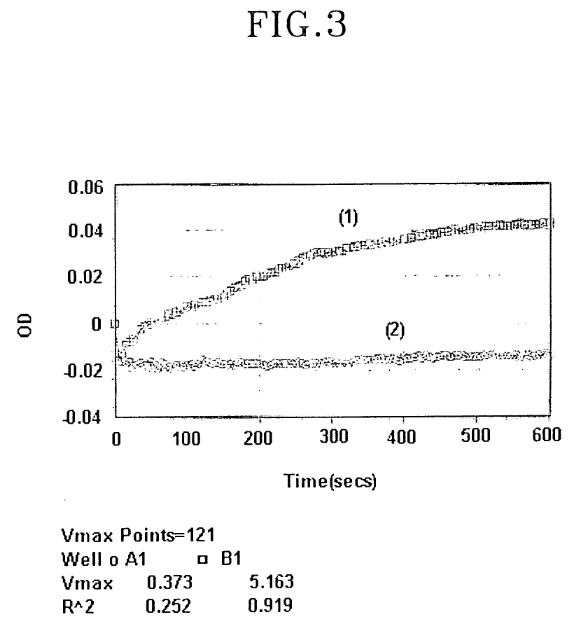

Then, each amplified PCR products were restricted with BamHI and KpnI and cloned into pCry1P-CMCase plasmid at the same restriction sites after excision of pre-cloned carboxymethylcellulase gene, and the cloned plasmids were transformed into Bacillus subtilis DB104 by natural transformation method (C. R. Harwood, et al., “Molecular Biological methods for Bacillus.” John Wiley & Sons, New York, p.416(1990)). The lipase activity was measured in the pure isolated spores after isolation of the spores from transformed Bacillus strain in the same manner as Example 1 (FIG. 3). In FIG. 3, the line (1) represents the spores displayed with wild type lipases and the line (2) is result of control spores, and the horizontal line refers to reaction time for lipase activity. These results indicate that proteins with hydrophobicity can be displayed on spore surface using the spore display system of this invention.

The lipases expressed in this Example contain their secretory signal peptides at N-terminus leading to extracellular secretion. The secreted enzymes can be attached on the surface of spore exposed to medium after sporulation. Furthermore, the non-secreted enzymes in spite of the presence of their secretory signal peptides (Bron S., J. Biotechnol., 64:3-13(1998)) also can be attached on the spore surface in the course of spore formation due to their hydrophobic property.

In a result, it is apparent that a protein with hydrophobicity can be displayed on spore surface regardless of that the protein is intracellularly expressed or secreted extracellularly.

### Example 3

### Display of Wild Type Carboxymethylcellulase on Spore Surface

For display of carboxymethylcellulase on spore surface, the carboxymethylcellulase gene isolated from Bacillus substilis BSE616 strain (Park S. H. et al., Agric. Biol. Chem., 55:441-448(1991)) was cloned under control of a promoter of cry1Aa toxin gene isolated from Bacillus thurigiensis strain.

At first, the cry1Aa promoter was amplified by PCR using primer 1AP1 (SEQ ID No:3) and primer 1AP2 (SEQ ID No;4) in the same condition as Example 2 with template of DNA isolated by Kalman S. et al. method (Appl. Environ. Microbiol., 59:1131-1137(1993)) from Bacillus thurigiensis kurstaki HD1 strain purchased from BGSC (Bacillus Genetic Stock Center, Ohio, USA). The PCR product was cloned into pGemT-easy vector purchased from Promega Co. (USA), subsequently digested with SphI and SalI and cloned into pUC19 plasmid (GenBank X02514) at the same restriction sites. The pUC19 plasmid was cleaved again with HindIII and BamHI restriction enzymes and the resultant fragment was inserted into pCPaC3 (KCTC 0831BP) at the same restriction sites, thereby constructing pCry1P-CMCase. The pCry1P-CMCase is a shuttle vector replicable both in E. coli and Bacillus strain.

The final constructed plasmid, pCry1P-CMCase, was transformed into Bacillus subtilis DB104 strain by natural transformation method. Other methods such as conjugation or transduction can be applied for introduction of the recombinant vectors into Bacillus strain. After then, the transformed Bacillus strain by pCry1P-CMCase was cultured for 24 hr at a shaking incubator (37□, 250 rpm) in GYS medium, and the only pure spores were isolated using renografin gradients method.

The measurement of carboxymetylcellulase activity in the isolated spores was carried out as follows: At first, 100□ of spore suspension in 0.1 M potassium phosphate (pH 6.0) at a density to be OD(600 nm)=1.4, was mixed with 200□ of 1% carboxymethylcellulose solution in 0.1 M potassium phosphate (pH 6.0), and reacted for 40 min at 50□. Following reaction, 900□ of DNS solution (20% potassium sodium tartarate, 1% NaOH, 0.05% NaHSO₃, 0.2% phenol, 1% 3,5-dinitrosalicyclic acid) was added to the reacted solution, heated for 5 min and chilled in cold water. Optical density of supernatant after centrifugation was measured at wavelength of 575 nm. The carboxymethylcellulase activity of enzymes displayed on spores surface was 1.96 mU compared to 0 mU in control.

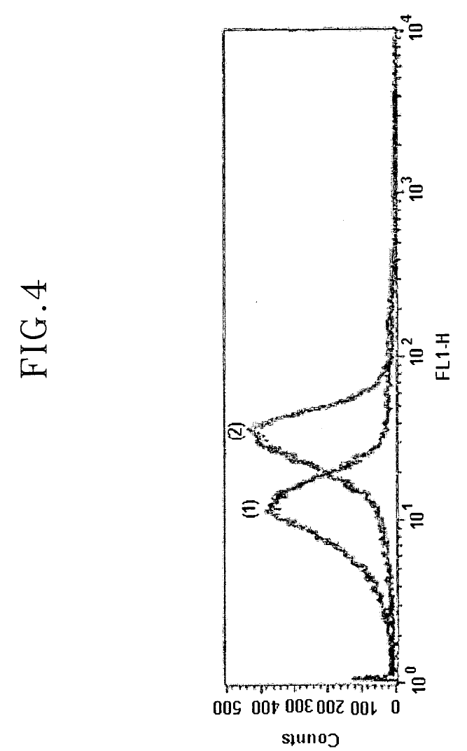

In another confirmation method, flow cytometry also showed the display of carboxymethylcellulase on the spore surface of Bacillus strain transformed with pCry1P-CMCase, when flow cytometry was performed by method of Kim et al. (Appl. Environ, Microbiol., 66:788-793(2000)) employing a carboxymethylcellulase specific antibodies (Kim et al., Appl. Environ, Microbiol., 66:788-793(2000)) and flow cytometer (FACSort, Becton Dickinson, USA) (FIG. 4). In FIG. 4, line 1 represents control spores, line 2 refers to spores of Bacillus strain transformed by pCry1P-CMCase, vertical line denotes the number of spores and horizontal line is the strength of fluorescence. As shown in FIG. 4, the peak is transferred to right part in the spores displaying carboxymethylcellulase compared to control, which indicates that much more antibodies specific to carboxymethylcellulase bind on the spore surface transformed with pCry1P-CMCase plasmid. In a result, carboxymethylcellulases are attached on the spore surface transformed with pCry1P-CMCase.

The carboxymethylcellulases expressed in this Example contain their secretory signal peptide at N-terminus leading to extracellular secretion. Although the secreted enzymes is likely to be attach on the surface of spore exposed to medium after sporulation, the non-secreted enzymes in spite of the presence of their secretory signal peptides (Bron S., J. Biotechnol., 64:3-13(1998)) also can be attached on the spore due to hydrophobic property of signal peptide. Consequently, it is understood that any protein with secretory signal can be displayed on spore surface by means of the spore-surface display system of the present invention.

### Example 4

### Spore Display of Carboxymethylcellulase with Modified Secretory Signal

N-terminal secretory signal peptide generally comprises N-terminal 2-3 cationic amino acid residues followed by hydrophobic domain and the cationic amino acids allow for the secretion of protein by binding to anionic phospholipid of cell membrane (Tjalsma H., Microbiol. Mol. Biol. Rev.,64:515-547(2000)). Furthermore, the substitution of the cationic amino acids with neutral ones is known to result in decrease of secretion (Chen M. and Nagarajan V., J. Bacteriol., 176:5796-5801(1994)). On the base of the facts, the inventors hypothesized that the decrease of protein secretion may increase the intracellular proteins and lead to much higher display of the proteins on spore surface owing to N-terminal hydrophobic domain of the proteins. In an effort to prove the hypothesis, the below experiment was carried out.

To clone the carboxymethylcellulase with secretory signal containing only hydrophobic domain without the cationic amino acid residues, DNA of Bacillus subtilis 168 strain (Nature, 390:249-256(1997)), gifted by Dr. F. Kunst of Pasteur Institute in France, was isolated by method of Kalman et al. Thereafter, PCR was performed using primer cmc-hp (SEQ No:5) and another primer (SEQ No:6) with template of the isolated DNA in a same condition as Example 2.

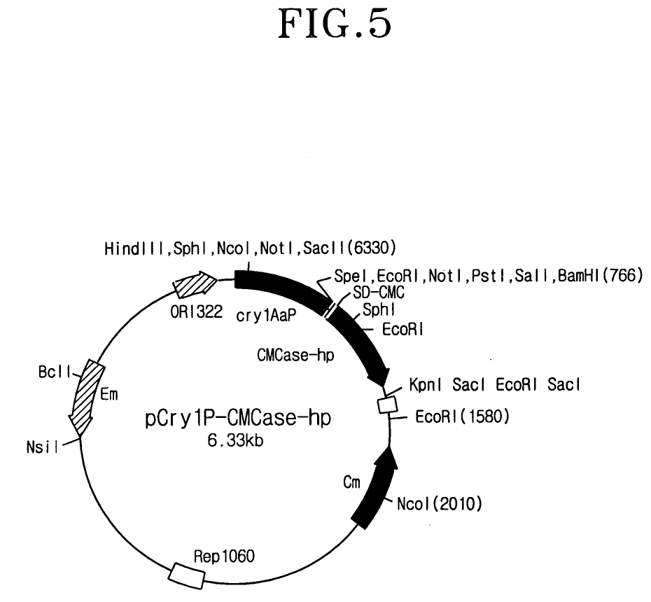

Subsequently, the PCR product was digested with BamHI and SacI, and cloned into the pCry1P-CMCase after excision of CMCase gene. The pCry1P-CMCase-hp (FIG. 5) was transformed into Bacillus subtilis DB104 by natural transformation method. The resulting transformant was denoted as “Bacillus subtilis BSK209”, deposited on Dec. 2, 2000 in International Depository Authority, the Korean Collection for Type Cultures (KCTC) and given accession number KCTC 0902BP. SEQ ID No:7 refers to the DNA sequence of the CMCase deficient of cationic amino acids in signal peptides and SEQ ID No:8 is amino acid sequence of the CMCase thereof.

And then, the transformed Bacillus strain BSK209 was cultured in a shaking incubator (37□, 250 rpm) and pure spores were isolated by renografin gradients method.

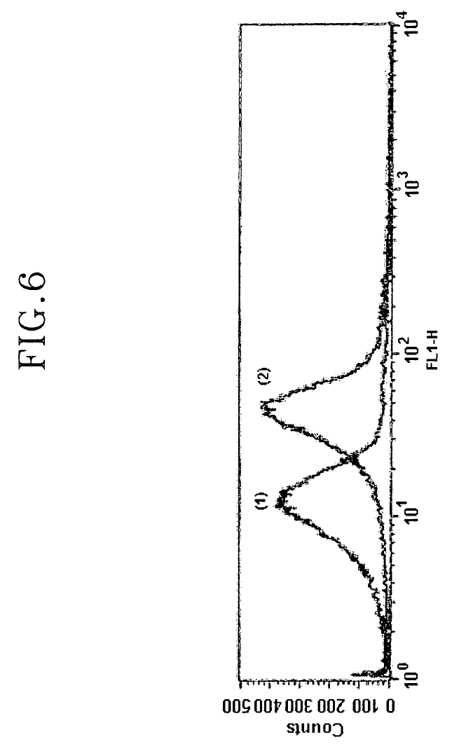

The carboxymethylcellulase activity of the isolated spores showed 4.74 mU compared to 0 mU of control under a same method as described in Example 3. This result is 2.4 times higher than that of wild type, and indicates that much more enzymes were displayed than wild type. Moreover, flow cytometry using carboxymethylcellulase specific antibodies in the same manner as Example 3, showed much more enzymes displayed on the spore surface of the Bacillus strain transformed with pCry1P-CMCase-hp (FIG. 6).

In FIG. 6, line 1 refers to control spore and line 2 is the spore of transformed Bacillus strain with pCry1P-CMCase-hp plasmid. As shown in FIG. 6, the peak is transferred to further right part compared to wild type carboxymethylcellulase shown in FIG. 4, which indicates much more carboxymethylcellulases bind to the spore surface. In a result, carboxymethylcellulase with secretory signal containing the only hydrophobic domain without the cationic residues is favorite form for spore display.

From these results, it is understood that deletion or neutralization of N-terminal cationic signal amino acids can be employed to gain further facility in spore display. Furthermore, it is apparent that fusion of hydrophobic domain of the signal peptides or other hydrophobic domain to protein of interest without its signal peptides can be used for spore display. In addition, it is also obvious that increase of hydrophobicity by selective or random mutagenesis of gene encoding surface protein of genetic carrier, or by fusion of other oligo- or polypeptides enhancing noncovalent linkage between carrier protein and protein of interest will enhance display of protein of interest on spore surface.

### Example 5

### Spore Surface Display of Carboxymethylcellulase Using Ionic Domain

Although spore surface of Bacillus is hydrophobic as shown in Example 1, there are also anionic ones (Nishihara T., et al., Microbiol. Immunol., 25:763-771(1981)). The present inventors hypothesized that a cationic protein of interest may be displayed by ionic bond and fusion of cationic motif into a protein of interest without cationic property will enable to display the protein of interest on spore surface. In an effort to verify the hypothesis, the below example was carried out.

In order to fuse a cationic domain to carboxymethylcellulase, 6 histidine residues were fused to N-terminus of mature form of the enzyme using primer as below. At first, PCR was performed using primer cmc-his (SEQ ID NO:9) and primer cmc-ter (SEQ ID NO:10) with template of DNA of Bacillus subtilis 168 in a same condition as Example 2.

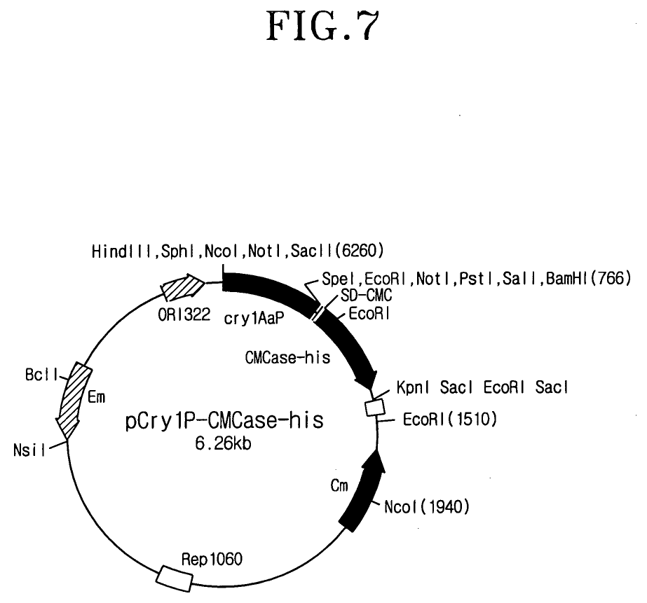

Subsequently, the PCR product was restricted with BamHI and SacI and cloned into pCry1P-CMCase described in Example 3 instead of CMCase gene. The constructed pCry1P-CMCase-his (FIG. 7) plasmid was transformed into Bacillus subtilis DB104 by natural transformation method. SEQ ID NO:11 refers to the gene sequence for CMCase fused with 6 histidine residues at its N-terminal region and SEQ No.12 is amino acid sequence thereof.

And then, the Bacillus strain transformed with pCry1P-CMCase-his was cultured in a shaking incubator (37□, 250 rpm) and pure spores were isolated by renografin gradients method.

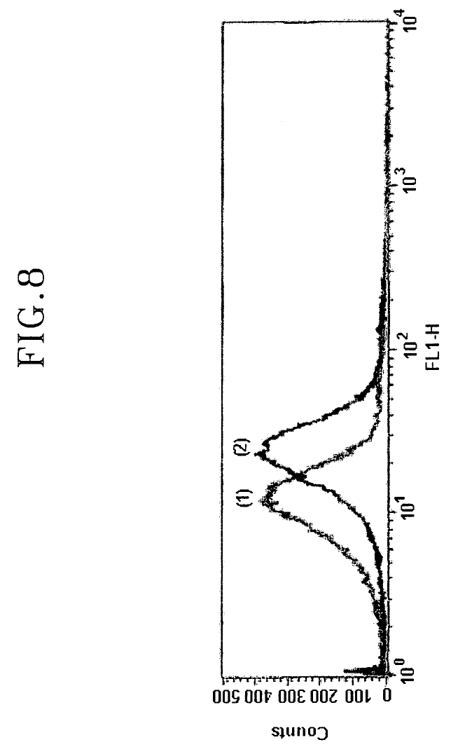

The carboxymethylcellulase activity of the isolated spores showed 1.90 mU compared to 0 mU of control under the same method as described in Example 3. And flow cytometry using carboxymethylcellulase-specific antibodies in a same manner as Example 3, showed enzymes displayed on the spore surface of the transformed Bacillus strain with pCry1P-CMCase-his (FIG. 8).

In FIG. 8, line 1 refers to control spore and line 2 represents the spore of transformed Bacillus strain with pCry1P-CMCase-his plasmid. As shown in FIG. 8, the peak is transferred to further right part compared to control, which indicates much more antibodies specific for carboxymethylcellulase bind to the spore surface. These results indicate that carboxymethylcellulase containing N-terminus with additional cationic domain bind to the spore surface of Bacillus transformed with pCry1P-CMCase-his.

Therefore, it is obvious that increase of cationic property by selective or random mutagenesis of gene or by fusion with cationic domain in any protein will enhance spore display. In addition, it is also apparent that the fusion of other ologo- or polypeptides enhancing noncovalent bond between surface protein of genetic carrier and protein of interest, or increase of anionic property by selective or random mutagenesis of gene encoding surface protein of genetic carrier will improve spore surface display of cationic protein of interest.

Based on the results of the Example showing increase of surface display by fusion of additional sequence, it is conceivable that it is helpful for surface display to fuse binding partners such as antibody-antigen or ligand-receptor to protein of interest and surface protein of genetic carrier.

Furthermore, it is understood that the protein of interest displayed will be more stabilized by the treatment of glutaraldehyde, UV or enzymes catalyzing formation of covalent bond, thereby forming additional covalent bond between surface protein of genetic carrier and protein of interest.

### Example 6

### Display of Protein of Interest on Phage Surface

On the facts and findings verified in the above examples, it becomes apparent that protein of interest capable of binding to coat proteins of phage can be displayed on phage surface. Such possibility was validated as follows: Moreover, it is very likely that a protein of interest incapable of binding to phage surface may be displayed on phage surface by fusing a motif capable of binding to coat protein.

At first, the hydrophobic domains are fused to both coat protein of phage and protein of interes. In addition, signal peptides for secretion toward periplasm are also fused. When they are expressed in a host cell, the protein of interest secreted is displayed on phage surface by hydrophobic interaction with coat protein of phage locating in periplasm.

A modification capable of displaying via other linkage other than hydrophobic interaction is apparent to those skilled in the art based on the findings in this Example.

### Example 7

### Directed Evolution of Protein of Interest Using Method for Display on Genetic Carrier

It is possible to carry out directed evolution of protein of interest using the display systems designed in the invention, in which surface display is enabled by interaction between protein of interest and surface of genetic carrier. At first, error prone PCR is performed with template of carboxymethylcellulase gene (Cadwell, R. C. and Joyce, G. F., PCR Methods Appl., 2:28-33(1992)). The PCR is performed using primers specific for carboxymethylcellulase gene with template of pCPaC3 plasmid described in Example 3.

PCR mixture is prepared by mixing 0.3 pM of each primers, 5 ng of DNA template, PCR solution (10 mM Tris(pH 8.3), 50 mM KCl, 7 mM MgCl₂, 0.01% (w/v) gelatin), 0.2 mM dGTP, 0.2 mM dATP, 1 mM dTTP, 1 mM dCTP, 0.15 mM MnCl₂, 5 U Taq polymerase from Bioneer (Korea) and DW up to 100□. Total 13 cycles of PCR are performed under condition of denaturation for 30 sec at 94□, annealing for 30 sec at 50□ and extension for 1 min at 721□.

Subsequently, the above PCR-amplified inserts are cloned into the replicable vectors and Bacillus substilis DB104 is transformed with the cloned vectors by natural transformation. And then, carboxymethylcellulas are displayed on spore surface by culturing the transformed Bacillus strain in a shaking incubator for 24 hr and pure spores are isolated by renografin gradients method. Thereafter, the spores with modified carboxymethylcellulase are selected using the change of carboxymethylcelluase activity or flow cytometry with antibody specific for carboxymethylcellulase as described in Example 3.

Example 8

### Bioconversion Using Genetic Carrier Surface-Displaying Protein of Interest

The bioconversion using lipase in organic solvent has been reported (Zaks, A. et al., Proc. Natl. Acad. Sci. USA. 82:3192(1985); and Klibanow, A. M., CHEMTECH, 16:354(1986)). It is indispensable to carry out reaction without inactivation of enzymes. To accomplish this purpose, fixation of lipases has been conventionally used (Mustranta, A. Forssell et al., Enz. Microb. Technol., 15: 133(1993); and Reetz, M. T. et al., J. Biotechnol. Biogen., 49:527(1996)). According to these reports, fixed lipases maintain high stability in organic solvent and increase synthesis compared to free lipases.

To begin with, lipases are displayed on surface of spore according to the present invention and bioconversion is performed as described method (Zaks, A. et al., Proc. Natl. Acad. Sci. USA. 82:3192(1985); Klibanow, A. M., CHEMTECH, 16;354(1986)).

The bioconversion according to the present invention can be also performed by displaying protein of interest on the surface of virus resistant to organic solvent.

### Example 9

### Protein Array Using Genetic Carrier Surface-Displaying on Protein of Interest

10⁶-10⁹ spores displaying monoclonal antibodies against specific surface antigen are attached onto glass substrate for protein array (BMS, Germany) with aldehyde functional group on its surface using automated array apparatus. The attachment is made in a form of covalent linkage, which is Schiff base between amino group of protein on spore surface and aldehyde group on surface of slide glass. Although the displayed proteins attached on solid surface may be inactivated, they may have orientation.

The protein array kit manufactured according to the present invention has a variety of applicable fields including diagnosis, analysis of gene expression, analysis of interaction between proteins, analysis of interaction between protein and ligand, study on metabolism, screening novel or improved enzymes, combinatorial biochemical synthesis and biosensor.

### Example 10

### Production of Antibody Genetic Carrier Surface-Displaying on Protein of Interest

Antibodies can be induced by displaying antigen capable to induce immune response in vivo.

Firstly, pCry1P-CMCase containing carboxymethylcellulase as antigen used in Example 3 is transformed into Bacillus subtilis DB104 by natural transformation method. And then, the carboxymethylcellulases are displayed on spore surface by culture the transformed Bacillus strain in a shaking incubator supplemented with GYS media for 24 hr, and subsequently pure spores are isolated by renografin gradients method. The antigen-displayed spores are resuspended into PBS, and same volume of adjuvant is added. Thereafter, the above solution is mixed by vortex and injected i.v. into BALB/c mouse with age of 6-8 wk after birth. After 4 wk, second injection is carried out. Antibodies are induced by additional 2-3 time boostings.

### Example 11

### Isolation of Specific Substance Using Genetic Carrier Surface-Displaying on Protein of Interest

It is possible to isolate specific substance from mixture using genetic carrier displaying binding domain. Firstly, error prone PCR is performed with template of gene encoding binding domain (Cadwell, R. C. and Joyce, G. F., PCR Methods Appl., 2:28-33(1992)). The PCR is performed using primers specific for gene of interest with template of plasmid containing gene of interest or chromosome. PCR mixture is prepared by mixing 0.3 μM of each primers, 5 ng of DNA template, PCR solution (10 mM Tris(pH 8.3), 50 mM KCl, 7 mM MgCl₂, 0.01% (w/v) gelatin), 0.2 mM dGTP, 0.2 mM DATP, 1 mM dTTP, 1 mM dCTP, 0.15 mM MnCl₂, 5 U Taq polymerase from Bioneer (Korea) and DW up to 100□. Total 13 cycles of PCR are performed under condition of denaturation for 30 sec at 94□, annealing for 30 sec at 50□ and extension for 1 min at 72□.

Subsequently, the above PCR-amplified inserts are cloned into the replicable vectors to construct library in host cells. The host cells are transformed with the cloned vector library, the binding domain is expressed in host cells and displayed on the surface of genetic carrier, and the carrier displaying modified binding domain with aimed property is screened. The screened genetic carrier is isolated, proliferated, expressed and used to isolate specific substance by mixing with the mixture.

As described in above, the present method for preparing protein of interest surface-displayed on genetic carrier can be applied to display various proteins on surface of genetic carrier in the absence of display motif, the full activity of protein of interest can be acquired owing to display after formation of its inherent structure, and the increase in an amount of protein of interest expressed and displayed never decreases a resistance against environment a viability of genetic carrier.

### REFERENCES

1. Agterberg, M., Adriaanse, H. and Tommassen, J. Use of the outer membrane protein PhoE as a carrier for the transport of a foreign antigenic determinant to the cell surface of Escherichia coli K-12. Gene 59:145-150(1987)

2. Agterberg, M., Adriaanse, H., van Bruggen, A., Karperien, M. and Tommassen, J. Outer membrane PhoE protein of Escherichia coli K-12 as an exposure vector: possibilities and limitations. Gene 88:37-45(1990)

3. Arnold, F. H. and Volkov, A. A. Directed evolution of biocatalysis. Curr. Opin. Chem. Biol. 3:54-59 (1999).

4. Charbit, A., Molla, A., Saurin, W. and Hofnung, M. Versatility of a vector for expressing foreign polypeptides at the surface of Gram-negative bacteria. Gene 70:181-189(1988)

5. Charbit, A., Sobczak, E., Michel, M.-L., Molla, A., Tiollais, P. and Hofnung, M. Presentation of two epitopes of the preS2 region of hepatitis B virus on live recombinant bacteria. J. Immunol 139:1644-1658(1987)

6. Chiswell, D. J. and McCafferty, J. Phage antibodies:will new ‘coliclonal’ antibodies replace monoclonal antibodies. TIBTECH 10:80-84(1992)

7. Daugherty, P. S., Chen G., Olsen, M. J., Iverson, b. L., Georgiou, G. Antibody affinity maturation using bacterial surface display. Protein Eng. 11:825-832 (1998)

8. d'Enfert, C., Ryter, A. and Pugsley, A. P. Cloning and expression in Escherichia coli of the Klebsiella pneumoniae genes for the production, cell surface localisation and secretion of the lipoprotein pullulanse. EMBO J. 6:3531-3538(1987)

9. Driks, A. Bacillus subtilis spore coat. Microbiol. Mol. Biol. Rev. 63(1):1-20 (1999)

10. Ferguson, M. A. J. and Williams, A. F. Cell-surface anchoring of proteins via glycosyl-phosphatidylinositol structures. Ann. Rev. Biochem. 57:285-320(1988)

11. Fischetti, V. A., Medaglini, D., Oggioni, M. and Pozzi, G. Expression of foreign proteins on Gram-positive commensal bacteria for mucosal vaccine delivery. Curr. Opin. Biotechnol. 4:603-610(1993)

12. Francisco, J. A., Earhart, C. F. and Georgiou, G. Transport and anchoring of b-lactamase to the external surface of Eshcherichia coli. Proc.Natl.Acad.Sci.USA 89:2713-2717(1992)

13. Freeman, A., Abramov, S., and Georgiou G. Site-protected fixation and immobilization of Escherichia coli cells displaying surface-anchored beta-lactamase. Biotechnol. Bioeng. 62(2) :155-159 (1999).

14. Fuchs, P., Breitling, F., Dubel, S., Seehaus, T. and Little, M. Targeting recombinant antibodies to the surface of Escherichia coli: fusion to a peptidoglycan associated lipoprotein. Bio/Technology 9:1369-1372(1991)

15. Georgiou, G. Analysis of large libraries of protein mutants using flow cytometry. Adv. Protein Chem. 55:293-315 (2000).

16. Georgiou, G., Poetschke, H. L., Stathopoulos, C. and Francisco, J. A. Practical applications of engineering Gram-negative bacterial cell surfaces. TIBTECH 11:6-10(1993)

17. Georgiou, G., Stathopoulos, C., Daugherty, P. S., Nayak, A. R., Iverson, B. L. and Curtiss III, R. Display of heterologous proteins on the surface of microorganisms: From the screening of combinatorial libraries to live recombinant vaccines. Nature Biotechnology 15:29-34(1997)

18. Georgiou, G., Stephens D., Stathopoulos, C., Poetschke H. L., Mendenhall J. and Earhart C. F. Display of b-lactamase on the Escherichia coli surface: outer membrane phenotypes conferred by Lpp′-OmpA′-b-lactamase fusions. Protein Eng. 9:239-247 (1996)

19. Hedegaard, L. and Klemm, P. Type 1 fimbriae of Escherichia coli as carriers of heterologous antigenic sequences. Gene 85:115-124(1989)

20. Jung, H. C., Lebeault, J. M. and Pan, J. G. Surface display of Zymomonas mobilis levansucrase by using the ice-nucleation protein of Pseudomonas syringae. Nature Biotechnol. 16:576-580 (1998a).

21. Jung, H. C., Park, J. H., Park, S. H., Lebeault, J. M. and Pan, J. G. Expression of carboxymethylcellulase on the surface of Escherichia coli using Pseudomonas syringae ice-nucleation protein. Enzyme Microb. Technol. 16:576-580 (1998b).

22. Kim, E. J. and Yoo, S. K. Cell surface display of CD8 ecto domain on Escherichia coli using ice-nucleation protein. Biotechnol. Tech. 12:197-201 (1998).

23. Kim, E. J. and Yoo, S. K. Cell surface display of hepatitis B virus surface antigen on Escherichia coli using Pseudomonas syringae ice-nucleation protein. Lett. Appl. Microbiol. 29:292-297 (1999).

24. Kim, Y. S., Jung, H. C. and Pan, J. G. Bacterial cell surface display of an enzyme library for selective screening of improved cellulase variants. Appl. Environ. Microbiol. 66:788-793 (2000).

25. Kwak, Y. D., Kim, E. J. and Yoo, S. K. Cell surface display of human immunodeficiency virus type I gp120 on Escherichia coli by using ice-nucleation protein. Clinic. Diag. Lab. Immun. 6:499-503 (1999).

26. Klauser, T., Kramer, J., Otzelberger, K., Pohlner, J. and Meyer, T. F. Characterization of the Neisseria Iga-core: The Essential unit for outer membrane targeting and extracellular protein secretion. J. Mol. Biol. 234:579-593(1993)

27. Klauser, T., Pohlner, J. and Meyer, T. F. Extracellular transport of cholera toxin B subunit using Neisseria IgA protease-domain: conformation-dependent outer membrane translocation. EMBO J. 9:1991-1999(1990)

28. Kornacker, M. G. and Pugsley, A. P. The normally periplasmic enzyme-lactamase is specifically and efficiently trnaslocated through the Escherichia coli outer membrane when it is fused to the cell-surface enzyme pullulase. Mol. Microbiol. 4(7):1101-1109(1990)

29. Lee, J. S., Shin, K. S., Pan, J. G. and Kim, C. J. Nature Biotechnol. 18:645-648 (2000)

30. Lewis P. J. and Errington J. Use of green fluorescent protein for detection of cell specific gene expression and subcellular protein localization during sporulation in Bacillus subtilis. Microbiology 142:733-740 (1996)

31. Little, M., Fuchs, P., Breitling, F. and Dubel, S. Bacterial surface presentation of proteins and peptides: an alternative to phage display technology, TIBTECH 11:3-5(1993)

32. Martineau, P., Charbit, A., Leclerc, C., Werts, C., O'Callaghan, D. and Hofnung, M. A genetic system to elicit and monitor antipeptide antibodies without peptide synthesis. Bio/Technology 9:170-172(1991)

33. Newton, S. M., Jacob, C. O. and Stocker, B. A. D. Immune response to cholera toxin epitope inserted in Salmonella flagellin. Science 244:70-72(1989)

34. Ochs, M., Angerer, A., Enz, S. and Braun, V. Surface signaling in transcriptional regulation of the ferric citrate transport system of Escherichia coli: mutational analysis of the alternative sigma factor FecI supports its essential role in fec transport gene transcription. Mol. Gen. Genet. 250:455-465 (1996)

35. Palva, A. M. and Palva, I. A. Lactobacillus expression system using surface protein gene sequences, W094/00581 (1994).

36. Richins, R. D., Kaneva, I., Mulchandani, A., and Chen, W. Biodegradation of organophosphorus pesticides by surface-displayed organophosphorus hydrolase. Nature Biotechnol. 15:984-987 (1997).

37. Samuelson, P., Hansson, M., Ahlborg, N., Androoni, C., Gotz, F., Bachi, T., Nguyen, T. N., Binz, H., Ulhen, M. and Stahl, S. Cell surface display of recombinant proteins on Stapholococcus carnosus. J. Bacteriol. 177(6):1470-1476 (1995).

38. Samuelson, P., Wernerus, H., Svedberg, M. and Stahl S. Staphylococcal surface display of metal-binding polyhistidyl peptides. Appl. Envir. Microbiol. 66: 1243-1248 (2000).

39. Schreuder, M. P., Mooren, A. T. A., Toschka, H. Y., Theo Verrips, C. and Klis, F. M. Immobilizing on the surface of yeast cells. TIBTECH 14:115-120 (1996).

40. Schulz, G. E. Bacterial porins: structure and function. Curr. Opin. Cell Biol. 5:701-707(1993)

41. Sleytr, U. B. and Sara, M. Bacterial and archaeal S-layer protein: structure-function relationships and their biotechnological applications. TIBTECH 15:1-9 (1997)

42. Stathopoulos, C., Georgiou G., and Earhart C. F. Characterization of Escherichia coli expressing an Lpp′OmpA(46-159)-PhoA fusion protein localized in the outer membrane. Appl. Microbiol. Biotechnol. 45(1-2):112-119 (1996).

43. Sousa, C., Cebolla, A., and de Lorenzo, V. Enhanced metalloadsorption of bacterial cells displaying poly-His peptides. Nature Biotechnol. 14:1017-1020 (1996).

44. Sousa, C., Kotrba, P., Ruml, T. Cebolla, A., and de Lorenzo, V. Metalloadsorption by Escherichia coli displaying yeast and mammalian metallothioneins anchored to the outer membrane protein LamB. J. Bacteriol. 180:2280-2284 (1998).

45. Taylor, I. M., Harrison, J. L., Timmis, K. N. and O'Conor, C. D. The TraT lipoprotein as a vehicule for the transport of foreign antigenic determinants to the cell surface of Escherichia coli K12 : structure-function relationships in the TraT protein. Mol. Microbiol. 4(8):1259-1268(1990)

46. Webb, C. D., Decatur A., Teleman A. and Losick R. Use of green fluorescent protein for ivsualization of cell specific gene expression and subcellular protein localization during sporulation in Bacillus subtilis J. Bacteriol. 177:5906-5911 (1995)

47. Zheng L and Losick R., Cascade regulation of spore coat gene expression in Bacillus subtilis J. Mol. Biol. 212: 645-660(1990)

What is claimed is:

1. A method for preparing a protein of interest surface-displayed on genetic carrier, which comprises the steps of:
(a) transforming a host cell harboring the genetic carrier selected from the group consisting of spore and virus with a vector containing a gene encoding the protein of interest;
(b) culturing the transformed host cell and expressing the protein of interest in the host cell; and
(c) allowing to form noncovalent bond between the expressed protein and a surface of the genetic carrier so that the protein of interest is displayed on the surface of the genetic carrier.

2. A method for improving a protein of interest, which comprises the steps of:
(a) constructing a gene library of the protein of interest by means of mutating the gene encoding the protein of interest;
(b) preparing a vector library containing the constructed gene library;
(c) transforming a host cell harboring a genetic carrier selected from the group consisting of spore and virus with the vector library;
(d) culturing the transformed host cell and expressing the variants of the protein of interest in the host cell;
(e) obtaining a genetic carrier library by means of allowing to form noncovalent bond between the expressed protein variant and a surface of the genetic carrier so that the variant is displayed on the surface of the genetic carrier; and
(f) screening the genetic carrier displaying on its surface the variant of the protein of interest having a desired property.

3. A method for isolating a substance of interest in mixture, which comprises the steps of:
(a) constructing a gene library encoding a variant of binding protein or binding domain thereof by means of mutating the gene encoding the binding protein or binding domain as protein of interest;
(b) preparing a vector library containing the constructed gene library;
(c) transforming a host cell harboring a genetic carrier selected from the group consisting of spore and virus with the vector library;
(d) culturing the transformed host cell and expressing the variants of the binding protein or binding domain in the host cell;
(e) obtaining a genetic carrier library by means of allowing to form noncovalent bond between the expressed binding protein variant or binding domain variant and a surface of the genetic carrier so that the variant is displayed on the surface of the genetic carrier;
(f) contacting the genetic carrier library with a predetermined substance and screening an improved binding protein or binding domain thereof by means of selecting the genetic carrier displaying on its surface the variant binding the predetermined substance;
(g) contacting the genetic carrier displaying on its surface the improved binding protein or binding domain thereof with the mixture to isolate the substance of interest in mixture.

4. The method according to claim 1 or 2, wherein the protein of interest is selected from the group consisting of hormone, hormone analogue, enzyme, enzyme inhibitor, signal transduction protein or fragment thereof, antibody or fragment thereof, single chain antibody, binding protein or fragment thereof, peptide, antigen, adhesive protein, structural protein, regulatory protein, toxin protein, cytokine, transcription regulatory protein, blood clotting protein and plant defense-inducing protein.

5. The method according to claim 3, wherein the binding protein or binding domain thereof is antibody or antibody domain thereof.

6. The method according to claim 4, wherein the binding protein or binding domain thereof is antibody or antibody domain thereof.

7. The method according to claim 3, wherein the binding protein or binding domain is selected from the group consisting of protease inhibitor, crambin, enterotoxin, conotoxin, apaminm lysozyme, ribonuclease, charybdotoxin, cystatin, eglin, ovomucoid, azurin, tumor necrosis factor and CD4.

8. The method according to claim 4, wherein the binding protein or binding domain is selected from the group consisting of protease inhibitor, crambin, enterotoxin, conotoxin, apaminm lysozyme, ribonuclease, charybdotoxin, cystatin, eglin, ovomucoid, azurin, tumor necrosis factor and CD4.

9. The method according to claim 3, wherein the binding protein is monomer or multimer.

10. The method according to claim 4, wherein the binding protein is monomer or multimer.

11. The method according to any one of claims 1-3, wherein the protein of interest is one modified to enhance noncovalent bond with the genetic carrier.

12. The method according to claim 11, wherein the protein of interest is modified by virtue of: (i) deleting a portion of amino acids of the protein of interest; (ii) fusing oligopeptide or polypeptide, which enhance noncovalent bond between the protein of interest and genetic carrier, to the protein of interest or deleted form of (i); (iii) subjecting the protein of interest to site-directed mutagenesis; or (iv) subjecting the protein of interest to random mutagenesis.

13. The method according to claim 12, wherein the deleting a portion of amino acids of the protein of interest is performed by deleting ionic amino acids from N-terminal sequence of the protein of interest.

14. The method according to claim 12, wherein the fused oligopeptide is cationic peptide.

15. The method according to any one of claims 1-3, wherein the genetic carrier has a surface protein modified to enhance noncovalent bond with the protein of interest.

16. The method according to claim 15, wherein the genetic carrier is modified by virtue of: (i) fusing oligopeptide or polypeptide, which enhance noncovalent bond between the protein of interest and genetic carrier, to the surface protein of genetic carrier; (ii) subjecting the surface protein of genetic carrier to site-directed mutagenesis; or (iii) subjecting the surface protein of genetic carrier to random mutagenesis.

17. The method according to any one of claims 1-3, wherein the host harboring spore is selected from the group consisting of a spore-forming Gram negative bacterium, a spore-forming Gram positive bacterium, a spore-forming Actionmycete, a spore-forming yeast and a spore-forming fungus.

18. The method according to claim 17, wherein the spore-forming Gram positive bacterium is selected from the group consisting of Clostridium, Paenibacillus and Bacillus.

19. The method according to claim 19, wherein the Bacillus is selected from the group consisting of Bacillus subtilis, Bacillus thuringiensis and Bacillus megaterium.

20. The method according to any one of claims 1-3, wherein the virus is a bacteriophage.

21. The method according to claim 20, wherein the bacteriophage is located in periplasm of host cell and the protein of interest is bound to a surface of the bacteriophage.

22. The method according to any one of claims 1-3, wherein the host cell is one mutated to eliminate a production of intracellular protease or extracellular protease which is involved in degradation of the surface-displayed protein of interest.

23. The method according to claim 2, wherein the step of screening is performed in such a manner that the spore library is treated with one or more selected from the group consisting of organic solvent, heat, acid, base, oxidant, dryness, surfactant and protease and then spore displaying on its surface the variant of protein of interest resistant to the treatment is selected.

24. The method according to claim 2, wherein the genetic carrier is spore and the step of screening is performed in such a manner that the spore library is primarily treated with one or more selected from the group consisting of organic solvent, heat, acid, base, oxidant, dryness and surfactant followed by secondary treatment with protease and then spore displaying on its surface the variant of protein of interest resistant to the protease is selected.

25. The method according to claim 1, wherein the method further comprises the step of screening the genetic carrier displaying on its surface the protein of interest.

26. The method according to claim 2, 3 or 25, wherein the step of screening is performed by means of (i) an activity of the protein of interest displayed on surface of genetic carrier; (ii) a protein being capable of recognizing a substance labeling the protein of interest; (iii) a labeled ligand being capable of binding to the protein of interest; or (iv) an antibody being capable of binding to the protein of interest specifically.

27. The method according to claim 26, wherein the screening by means of a labeled ligand being capable of binding to the protein of interest or an antibody being capable of binding to the protein of interest specifically is performed by virtue of flow cytometry.

28. The method according to any one of claims 1-3, wherein the method further comprises the step of stabilizing the bond between the surface of the genetic carrier and the protein of interest by means of forming covalent bonds to between the surface of the genetic carrier and the protein of interest by use of physical, chemical or biochemical methods following displaying the protein of interest on the surface of genetic carrier via noncovalent bond.

29. The method according to claim 28, wherein the chemical method to form covalent bond is a treatment of glutaraldehyde, the physical method is a treatment of ultraviolet and the biochemical method is a treatment of enzyme ensuring formation of covalent bond.

30. A vector for displaying on surface of genetic carrier a protein of interest, which comprises a replication origin, an antibiotic-resistance gene, a restriction site, a gene encoding the protein of interest, wherein the protein of interest, when expressed in host cell, is capable of forming noncovalent bond to the surface of genetic carrier.

31. The vector according to claim 30, wherein the gene encoding the protein of interest is one mutated to enhance noncovalent bond between the surface of genetic carrier and the protein of interest.

32. The vector according to claim 31, wherein the gene encoding the protein of interest is mutated to (i) delete a portion of amino acids of the protein of interest; (ii) fuse oligopeptide or polypeptide, which enhance noncovalent bond between the protein of interest and genetic carrier, to the protein of interest or deleted form of (i); (iii) subject the protein of interest to site-directed mutagenesis; or (iv) subject the protein of interest to random mutagenesis.

33. A microbial transformant, characterized in that the transformant is produced by transformation a host cell harboring spore or virus with a vector according to any one of claims 30-32.

34. The transformant according to claim 33, wherein the host cell is one mutated to eliminate a production of intracellular protease or extracellular protease which is involve in degradation of the surface-displayed protein of interest.

35. A complex between genetic carrier and protein of interest, characterized in that the complex is prepared by displaying on the surface of the genetic carrier, according to the method of claim 1, hormone, hormone analogue, enzyme, enzyme inhibitor, signal transduction protein or fragment thereof, antibody or fragment thereof, single chain antibody, binding protein or fragment thereof, peptide, antigen, adhesive protein, structural protein, regulatory protein, toxin protein, cytokine, transcription regulatory protein, blood clotting protein or plant defense-inducing protein.

36. The complex according to claim 35, wherein the protein of interest is one modified by virtue of: (i) deleting a portion of amino acids of the protein of interest; (ii) fusing oligopeptide or polypeptide, which enhance noncovalent bond between the protein of interest and genetic carrier, to the protein of interest or deleted form of (i); (iii) subjecting the protein of interest to site-directed mutagenesis; or (iv) subjecting the protein of interest to random mutagenesis.

37. The complex according to claim 35, wherein the complex has additional covalent bonds to stabilize the bond between the surface of the genetic carrier and the protein of interest, in which the covalent bonds are formed by use of physical, chemical or biochemical methods following displaying the protein of interest on the surface of genetic carrier via noncovalent bond.

38. The complex according to any one of claims 35-37, wherein the genetic carrier is a spore.

39. The complex according to claim 38, wherein the spore is non-reproductive one obtained by means of one or more methods selected from the group consisting of genetic method, chemical method and physical method.

40. The complex according to claim 39, wherein the genetic method to make the spore non-reproductive is performed by deleting a gene involved in spore reproduction of host cell.

41. The complex according to claim 38, wherein the spore is derived from a variant mutated to increase its agglutination property by one or more methods selected from the group consisting of genetic method, chemical method and physical method.

42. The complex according to claim 35, wherein the protein of interest is monomer or multimer.

43. The complex according to any one of claims 35-37, wherein the genetic carrier is a bacteriophage.

44. A genetic carrier library displaying on its surface variants of a protein of interest, prepared by a process comprising the steps of: (a) constructing a gene library of the protein of interest by means of mutating the gene encoding the protein of interest; (b) preparing a vector library containing the constructed gene library; (c) transforming a host cell harboring a genetic carrier selected from the group consisting of spore and virus with the vector library; (d) culturing the transformed host cell and expressing the variants of the protein of interest in the host cell; (e) obtaining a genetic carrier library by means of allowing to form noncovalent bond between the expressed protein variant and a surface of the genetic carrier so that the variant is displayed on the surface of the genetic carrier; and (f) screening the genetic carrier displaying on its surface the variant of the protein of interest having a desired property.

45. The genetic carrier library according to claim 44, wherein the genetic carrier is a spore.

46. The genetic carrier library according to claim 44, wherein the genetic carrier is a bacteriophage and the variants of the protein of interest is variants of binding protein or binding domain.

47. A method for bioconversion using protein with activity for conversion reaction, characterized in that the method employs the complex between genetic carrier and protein of interest according to any one of claims 35-37.

48. The method according to claim 47, wherein the protein having activity for conversion reaction is enzyme or antibody.

49. A method producing an antibody to antigen in vertebrates, characterized in that the method comprises administering to vertebrates a composition containing an immunologically effective amount of the complex between genetic carrier and protein of interest according to any one of claims 35-37.

50. A protein microarray comprising a solid substrate and a material immobilized onto the substrate, characterized in that the material immobilized onto the substrate is selected from the group consisting of the complex between genetic carrier and protein of interest according to any one of claims 35-37; and the genetic carrier library according to any one of claims 44-46.
